(19) United States
(12) Patent Application Publication
(10) Pub. No.: US 2007/0156450 A1
(43) Pub. Date: Jul. 5, 2007

(54) Networked modular and remotely configurable system and method of remotely monitoring patient healthcare characteristics

(76) Inventors: Steven Roehm, Waukesha, WI (US); Ray Liu, Milwaukee, WI (US); Gopal B. Avinash, Waukesha, WI (US); Bruce Friedman, Tampa, FL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/324,852

(22) Filed: Jan. 4, 2006

(51) Int. Cl. G06Q 10/00; A61B 5/00

(52) U.S. Cl. 705/2; 600/300

(57) ABSTRACT

The present invention is a system and method of remotely monitoring patient health care characteristics. The system utilizes at least two micro-scale to millimeter-scale sensors, a wireless network, a central hub and pre-processing center and a means for notifying a clinician of the remote patient's condition. The system and method includes sensor to sensor coordination, modular-based sensors and processing, and allows a clinician to remotely configure the system.

### FIELD OF THE INVENTION

The invention is related to the field of patient monitoring. More specifically, the invention is related to the field of remote patient monitoring.

### BACKGROUND OF THE INVENTION

The ability to remotely monitor patient health characteristics greatly expands the scope of services possible for medical diagnostics and treatment. Traditional patient monitoring often requires bulky and expensive equipment specialized for each sensing parameter, so the sensing and monitoring are often limited to the hospital or clinic. Current miniaturization of this equipment has allowed for the development of mobile sensing devices, and advancements in communication technology have allowed for such devices to be monitored remotely, such as in a patient's home. This combination of the technological advancements is known as remote patient monitoring.

While remote patient monitoring currently exists to some extent, its full capabilities have yet to be realized. Specifically, most sensing devices are able to monitor only one parameter at a time, and the information is directly sent to the monitoring center. Different sensors do not have an effective system to communicate with each other, and the processing of sensed information cannot be performed in real time with integrated information from all sensors. The result of this incompatibility is a limited set of sensing applications and a sensing system that is not adaptable to changing needs of the patient.

In addition, current sensors for remote patient monitoring are still too bulky for very portable applications, and they are costly such that they are not disposable. The sensors that exist in remote patient monitoring systems today are also not autonomous, meaning that the patient must proactively turn on the sensor or apply the sensor every time information is collected.

### SUMMARY OF THE INVENTION

The present invention is a system and method of remotely monitoring patient health care characteristics. The system utilizes at least two micro scale to millimeter-scale sensors, a wireless network, a central hub and pre-processing center and a means for notifying a clinician of the remote patient's condition. The system and method includes sensor to sensor coordination, modular-based sensors and processing, and allows a clinician to remotely configure the system.

In one aspect of the present invention, a modular sensing system for remotely monitoring patient healthcare characteristics comprises a plurality of sensors configured in a home environment of a patient, and further configured to collect a set of patient health characteristics from a patient, wherein each of the plurality of sensors is capable of sensing multiple parameters, a central hub configured in the home environment, wherein the central hub receives the set of patient health characteristics, whereby the central hub is also configured to process the set of patent health characteristics and output a filtered set of patient health characteristics, a wireless network configured to couple the plurality of sensors and the central hub, wherein each of the plurality of sensors is configured to continuously communicate with each other and with the central hub, wherein each of the plurality of sensors is autonomous, thereby capable of automatic configuration with the modular sensing system.

The plurality of sensors are in a range from micro-scale to millimeter-scale, wherein the plurality of sensors may be any of a number of sensor types including: invasive implantable; non-invasive, portable device embeddable; and, non-invasive, home device embeddable, and further wherein each of the plurality of sensors are automatically configurable in real time and the plurality of sensors are portable and disposable. The central hub is further configured to evaluate the set of patient health characteristics as best, comparative or cumulative. Each of the plurality of sensors may be directly coupled with the central hub through the wireless network or with the central hub through the wireless network in an ad-hoc fashion. The present invention further comprises of a sensing sub-system configured to receive the set of patient health characteristics from the plurality of sensors, and further configured to relay the set of patient health characteristics to the central hub. A clinician hub coupled with the central hub, the clinician hub configured to receive a patient status and the filtered set of patient health characteristics from the central hub and to send the patient status to an appropriate clinician. The central hub is configured to integrate multiple sensing parameters and multiple sensing applications.

Another aspect of the present invention is a method of remotely monitoring patient healthcare characteristics, the method comprising collecting a set of patient healthcare characteristics from a patient with a plurality of sensors, processing the set of patient healthcare characteristics in a central hub to produce a filtered set of patient health characteristics, wherein the plurality of sensors and the central hub are configured in a home environment, and further wherein the plurality of sensors sends a patient status and the filtered set of patient health characteristics from the central hub to a clinician hub, directing the patient status and the filtered set of patient health characteristics with the clinician hub to an appropriate clinician and accessing the patient status by the clinician with a graphical user interface. The size of the plurality of sensors are in a range from micro-scale to millimeter-scale and the plurality of sensors may be any of a number of sensor types including: invasive implantable; non-invasive, portable device embeddable; and, non-invasive, home device embeddable.

The clinician hub may be configured outside of the home environment. The patient status includes a report when the set of patient health characteristics is within a predetermined normal range, and the patient status includes the report and an alarm when the set of patient health characteristics are not in the predetermined normal range. The central hub is configured to evaluate the set of patient health characteristics as best, comparative or cumulative with the central hub and each of the plurality of sensors is directly coupled with the central hub through the wireless network. The present invention integrates multiple sensing parameters and multiple sensing applications in the central hub.

A further aspect of the present invention is a system for remotely monitoring patient healthcare characteristics, the system comprising a plurality of sensors configured in a home environment of a patient, and further configured to collect a set of patient health characteristics from a patient, a central hub configured in the home environment, wherein the central hub receives the set of patient health characteristics, whereby the central hub is also configured to proceed the set of patient health characteristics and output a filtered set of patient health characteristics, a wireless network configured to couple the plurality of sensors and the central hub, a clinician hub coupled with the central hub, the clinician hub configured to receive a patient status and the filtered set of patient health characteristics from the central hub and to send the patient status to an appropriate clinician, and a graphical user interface configured such that the clinician can access the clinician hub and reconfigure the central hub. The size of the plurality of sensors are in a range from micro-scale to millimeter-scale and the plurality of sensors may be any of a number of sensor types including: invasive implantable; non-invasive, portable device embeddable; and, non-invasive, home device embeddable.

The clinician hub of the present invention may be configured outside of the home environment. The patient status of the present invention includes a report when the set of patient health characteristics is within a predetermined normal range, and the patient status includes the report and an alarm when the set of patient health characteristics are not in the predetermined normal range. The alarm is also sent to the patient and the central hub is further configured to evaluate the set of patient health characteristics as best, comparative or cumulative. The plurality of sensors is directly coupled with the central hub through the wireless network. This system also has a plurality of sensors that are coupled with the central hub through the wireless network in an ad-hoc fashion, further comprising a sensing sub-system configured to receive the set of patient health characteristics from the plurality of sensors, and further configured to relay the set of patient health characteristics to the central hub. The clinician reconfigures the central hub to alter the set of patient health characteristics collected by the plurality of sensors. The central hub is configured to integrate multiple sensing parameters and multiple sensing applications.

### BRIEF DESCRIPTION OF THE DRAWINGS

FIG. 1 illustrates a block diagram of an embodiment of the system of the present invention.

FIG. 2 illustrates a block diagram of an embodiment of the method of the present invention.

FIGS. 3a-3b illustrate a block diagram of an embodiment of the central hub of the present invention.

FIG. 4 illustrates a block diagram of an embodiment of the central hub of the present invention.

FIGS. 5a-5b illustrate a block diagram of an embodiment of the central hub of the present invention.

FIG. 6 illustrates a block diagram of an embodiment of the system of the present invention.

FIG. 7a-7c illustrate a flow chart of an embodiment of the method of the present invention.

FIG. 8 illustrates a block diagram of an embodiment of the home environment system of the present invention.

FIGS. 9a-9c illustrate a block diagram of an embodiment of the system of the present invention.

FIG. 10 illustrates a block diagram of an embodiment of a graphical user interface of the present invention.

### DETAILED DESCRIPTION OF THE INVENTION

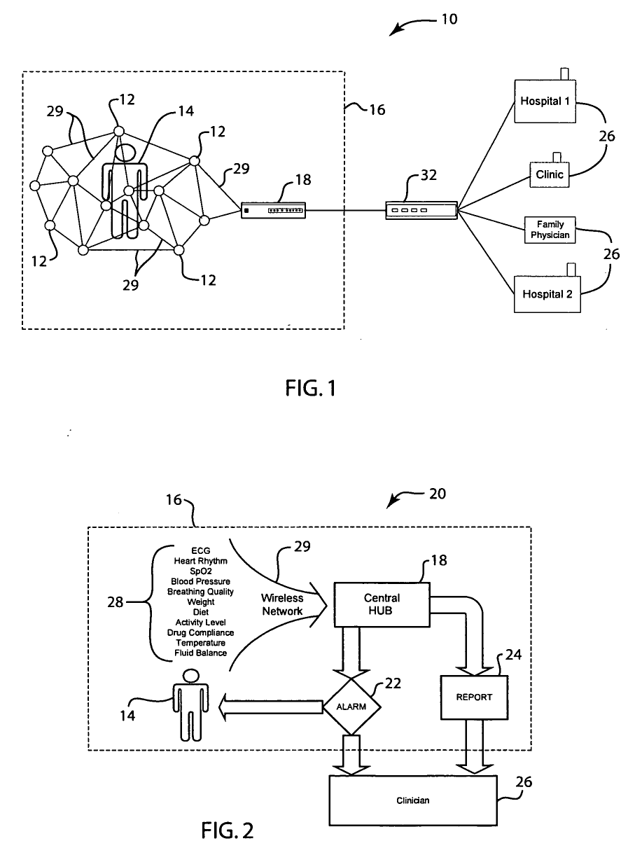

A block diagram of an embodiment the remote patient monitoring system 10 is shown in FIG. 1. The system 10 includes at least two sensors 12 to collect information from a patient 14 in a home environment 16 of the patient 14, but may have significantly more than two sensors 12. The multiple sensors 12 may collect the same information parameter from a different patient 14, different information parameters from the same patient 14, or different information parameters from different patients 14. The remote patient monitoring system 10 described may also include multiple sensing parameters, invasively implantable patient sensors 12, non-invasive embeddable, wearable sensors 12, as well as non-invasive embeddable, portable device or home device sensors 12.

The sensors 12 are preferably integrated into micro-scale to millimeter-scale devices (or smaller). Such small-scale sensors 12 bring many advantages and allow a host of new applications. One such advantage is environment sensing, wherein an indefinite number of these micro-sensors 12 are scattered into the environment to detect parameters or perturbations. Such small sensors 12 can be nearly invisible to the naked eye, and therefore do not disturb the home environment 16 in any way. Another advantage is wearable sensing, in that micro-scale sensors-12 can be applied to the surface of the patient 14 without notice to the patient 14. Yet another advantage is implantable sensing, wherein a micro-scale sensor 12 can be implanted into the patient 14 without harm or impact to the patient, and embeddable sensing, wherein a micro-scale sensor 12 can be embedded into many objects such as, but not limited to, clothing, appliances, furniture, portable devices, glasses, electronics, and home fixtures. Each sensor 12 can be embedded into a portable device of the patient 14, such as, but not limited to, a cane or walking stick, a keychain, remote control, furniture, or appliance.

In a preferred embodiment, the sensors 12 are omni sensing in that they integrate multiple sensors 12, sensing multiple parameters and data types. These sensors 12 are configured to sustain continuous communication and coordination between multiple sensors 12 and/or the central hub 18 in realtime, such that the processing of any sensed information can be performed in real time, and integrated with any other information from other sensor systems. This effective communication allows the system 10 to automatically adjust in real time. Specifically, the system 10 is able to turn specific sensors 12 on and off, modify the sensing parameters, change sensor 12 sensitivities, as well as save system 10 power, and any other system function related to the sensors 12.

Still referring to FIG. 1, the sensors 12 in the home environment 16, and the central hub 18 are constructed such that they are portable, and the sensors 12 are constructed to be disposable as well. The portable and disposable nature of the sensors 12 allows a system 10 that includes sensors 12 that are scattered in the home environment 16. This allows for relative ease in expanding or reducing the size of the home environment 16 by merely scattering new sensors 12, for example, in an additional room of a home, in an automobile of the patient, or in the patient's 14 workspace in addition to their home. Likewise, these portable and disposable sensors 12 are easily collected or deactivated in order to reduce the size of the home environment 16. Sensors that are currently used in the art of remote patient monitoring are much too bulky to be “dusted” in such a way, and are too costly to be disposable. In addition, the sensors 12 are designed to be autonomous, such that they can automatically configure themselves with the rest of the sensor system 10. In other words, a new sensor 12 added to the system 10 will immediately communicate and coordinate with the other sensors 12 in the system 10 in order to detect what the other sensors 12 are sensing and whether that new sensor 12 should be activated or deactivated. In current remote patient monitoring systems, the patient must proactively turn on the sensor or apply the sensor every time information is collected, and often times, a single sensor is only intended and able to sense one particular sensing parameter.

In additional embodiments of the present invention, each new sensor 12 module comes with a software upgrade pack to automatically upload new processing capabilities into the central hub 18. Thus, the software platform of the central hub 18 is also modular-based, such that new upgrades can integrate with the existing features to enable a host of additional processing capabilities that utilize the integrated information. Thus, as soon as a new module is plugged into the system, the system automatically upgrades its overall processing capabilities to include the enhanced capabilities of the new module.

A method of notifying 20 a clinician 26 of the remote patient condition is depicted in FIG. 2. This can be done either through an alarm 22 or a report 24, as shown in FIG. 2. The system 10 provides an interface for this information to be viewed by the clinician 26, which will be discussed later. This interface may also allow the clinician 26 to interact with the information, such as to provide a response to the patient 14. Referring again to FIG. 2, a set of patient health characteristics 28 is collected from the patient 14 and transferred over a wireless network 29 to a central hub 18 for pre-processing. The central hub 18 is configured to report 24 to the clinician 26 when the central hub 18 receives and processes a set of patient heath characteristics 28. In the case where the central hub 18 receivers characteristics 28 outside a predetermined acceptable range for the patient 14, the alarm 22 will alert the clinician 26, as well as the patient.

The system and method allows for the sensors to collect data for multiple sensing parameters at the same time, including such parameters as, but not limited to, ECG data, heart rhythm, partial pressure of oxygen, blood pressure, breathing quality, temperature, weight, activity levels, drug compliance, hydration and sleep habits. This information can then be integrated for analysis at the central hub. Table 1 illustrates particular sensors 12 in the system 10, their associated acquisition parameters, and whether those sensors 12 are configured to sense continuously for that acquisition parameter.

TABLE 1

| Sensor | Acquisition Parameter | Continuous? |
|---|---|---|
| ECG | waveform | yes |
| Heart rhythm | heart rate/pulse rate | yes |
| SpO₂ | partial pressure/oxygen saturation | yes |
| Blood Pressure | blood pressure | no |
| Breathing quality | breath rate/volume | no |
| Weight | kg | no |
| Activity Level | movement | yes |
| Drug Compliance | drug | no |
| Sleep Habits | multi-parameter (HR, breathing, etc.) | yes |
| Temperature | temperature | no |
| Fluid Balance | fluid concentration | yes |

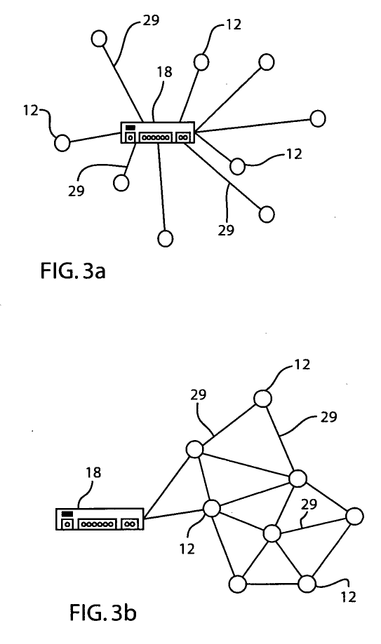

The system 10 includes an established wireless network whereby each sensor 12 is connected with each other wirelessly and is configured to communicate through the wireless network as depicted in FIGS. 3a & 3b. Each sensor 12 is also able to communicate to a central hub 18 through this wireless network 29. The network 29 may be configured where all sensors 12 connect and communicate directly with the hub 18, or it may be configured as an ad-hoc network 29 where each sensor 12 connects with another sensor 12 that bridges the connection to the central hub, as in FIG. 3b.

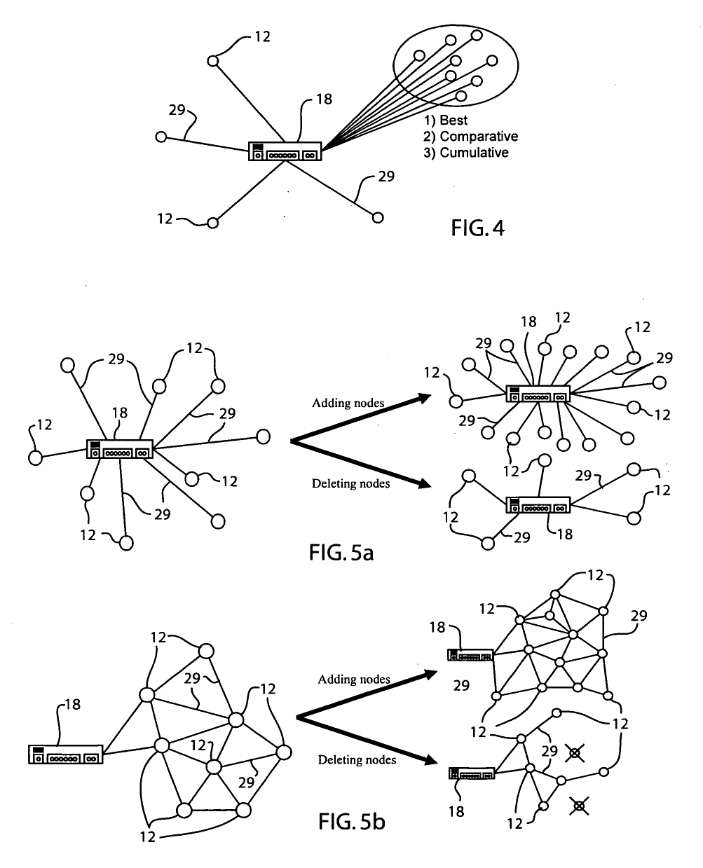

The system 10 consists of a central hub 18 or pre-processing center, whereby collected information from multiple sensors 12 is integrated and processed as shown in FIG. 4. This central hub 18 also provides an interface to either receive commands from the patient 14, or to send signals and/or directions to the patient 14. The pre-processing center 18 is also able to evaluate the data, e.g. as best or most relevant, comparative, or cumulative, as shown, and perform operations on the sensed data. The result of the processing may trigger an action, such as, but not limited to, an alarm to the patient 14, an alert to the clinician 26, a treatment to the patient 14, a reminder to the patient 14, a report to the patient 14, a report to the clinician 26 or a report to an insurance agency.

Multiple sensors 12 are able to coordinate with each other in an automated ad-hoc fashion to collect the relevant data for the relevant parameters. This includes the capability to turn on and off sensors 12, modify the sensing parameters, modify the sensor 12 sensitivity, or reconfigure the sensors 12. The sensors 12 continuously communicate with each other, and automatically establish communications connections with each other, as well as with the central hub 18, as long as the distance is within communication range. Therefore, new connections can be established with the addition of new sensors 12 to the system 10, or old connections can be deleted with the removal of old sensors 12 from the system 10. FIG. 5a shows the addition and removal of sensors 12 and the resulting communication connections with the central hub 18. FIG. 5b shows the addition and removal of sensors and the resulting communication connections with other sensors 12.

Each sensor 12 can be considered a module, whereby it can be added or removed from the system 10 without compromising the performance of the other sensors 12 in the system 10. When a new sensor 12 is added, it can be integrated into the existing network 14 and effectively perform its sensing functions in an integrated fashion with the other sensors 12. Again, FIGS. 5a and 5b show the configurations for adding and removing sensors 12 to both the central hub 18 and the existing ad-hoc sensor 12 network 14. Examples of sensors 12 that can be added or deleted to the system 10 include blood glucose monitoring, pulse oximetry monitoring, heart rate monitoring, cardiac signal monitoring, brain signal monitoring, breathing quality monitoring, fluid balance monitoring, activity level monitoring, drug compliance monitoring, sleep monitoring and diet monitoring sensors.

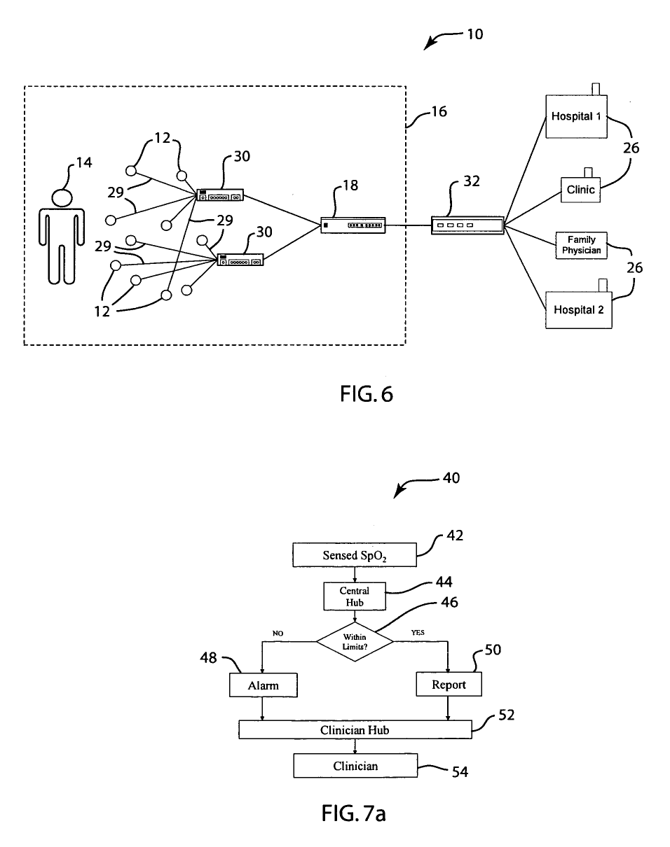

The system and method of the present invention also allows the physician or clinician to remotely configure or reconfigure the parameters of the sensing system 10 such that on-demand medical diagnostics can be achieved from a remote location. A block diagram of an alternative embodiment of the remote patient monitoring system 10 is shown in FIG. 6. The minimum requirements for realizing such a system 10 can be seen through the four basic components, including the sensors 12 used for gathering the physiological parameters from the patient 14. As stated previously, these parameters may include, but are not limited to, ECG signals, heart rhythm, partial pressure of oxygen, blood pressure, breathing quality, weight, temperature, activity levels, drug compliance, sleep habits, and fluid balance. The second basic component is the sensing sub-system 30, used for integrating the sensed information from its associated sensors 12 for a given function or application. The third basic component is the central hub 18, used for integrating and processing the data received by the sensing sub-systems 30. This component allows for multiple sub-systems 30 to be processed together for creating new and configurable applications. The central hub 18 also provides an interface for the patient 14 to interact or communicate with the clinician 26. The central hub 18 component is also used for storing the sensed data and processed results. The fourth basic component is the clinician hub 32, used for routing the sensed data and processed results to the respective clinicians 26 as necessary. This unit is also used as an access point that provides an interface for clinicians 26 to connect with the home environments 16 being monitored. Through this interface, the clinician 26 is able to access the home to reconfigure the sensing applications being monitored.

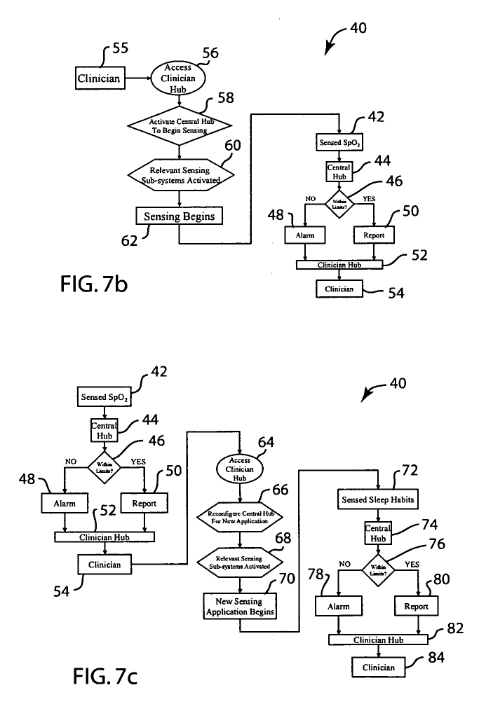

The method 40 of the present invention is illustrated in FIGS. 7a-7c. In FIG. 7a, a pulse oximeter is used to remotely sense the saturation of oxygen in the patient's hemoglobin and send the data to the clinician. The method 40 is continuously monitoring the patient, and data is being sent to the clinician in the form of a report. Still referring to FIG. 7a, the method 40 of the present invention senses the oxygen saturation of the patient in step 42. In step 44, the central hub receives the oxygen saturate data, and in step 46, it is determined whether the oxygen saturation is within acceptable limits. If the oxygen saturation is within acceptable limits, then a report is generated in step 50 and sent to the clinician hub in step 52. If the oxygen saturation is not within acceptable limits, then in step 48 an alarm is sent to the clinician hub in step 52. In either case, the report from step 50 or the alarm from step 48, when sent to the clinician hub in step 52, is subsequently passed on to the clinician in step 54.

In FIG. 7b, the clinician initiates the method 40 to begin monitoring by accessing the interface at the clinician hub and selecting the desired sensing configurations of the central hub to activate. The clinician initiates the method in step 55. In step 56, the clinician accesses the clinician hub, and in step 58 the clinician activates the central hub through the clinician hub to begin sensing. In step 60, the activation causes the relevant sensing subsystems to be activated, and step 62 the sensing begins. The steps 42-54 of FIG. 7b have been previously described in the description for FIG. 7a.

In FIG. 7c, the clinician accesses a monitoring system in order to re-configure it to better suit the changing condition of the patient, either by adding a new sensing application, removing an existing application, or re-defining the sensitivity parameters of an application. Referring now FIG. 7c, after the clinician receives, in step 54, the alarm from step 48 or the report from step 50 through the clinician hub in step 52, the clinician accesses the clinician hub in step 64. Thereafter, the clinician reconfigures the central hub for a new application in step 66. In step 68, the relevant sensing subsystems are activated, and in step 70 the new sensing application begins. In this example, sensed sleep habits are detected in step 72, and sent to the central hub in step 74. It is then determined in step 76 whether the sensed sleep habits fall within acceptable limits, and if they do, then in step 80 a report is generated. If the sensed sleep habits do not fall within acceptable limits then an alarm is generated in step 78. In either case, the report from step 80 or the alarm from step 78 are sent to the clinician hub in step 82, wherein they can be accessed by the clinician in step 84. While the example shown here is for the case of a patient being located in a home environment, the remote patient monitoring method 40 can be applied for any remote sensing scenario, including within the hospital. It should also be noted that the Sp0₂ sensing in step 42 and the sleep habit sensing in step 72 are exemplary and any sensing parameter may be monitored, reported and selected utilizing this method.

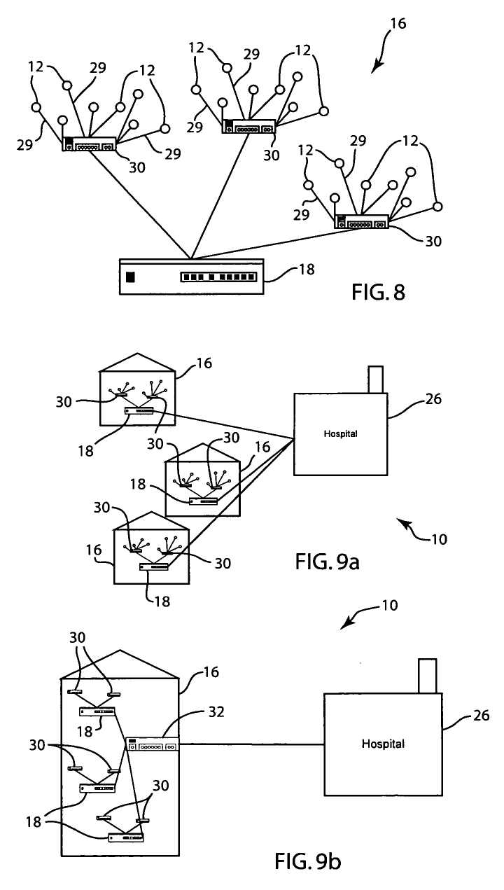

Referring now to FIG. 8, the system 10 of the preferred embodiment includes a home environment 16 with a central hub 18, whereby collected information from multiple sensing sub-systems 30 is integrated and processed. The central hub 18 is able to evaluate and perform operations on the sensed data from the sensing sub-systems 30. The central hub 18 is configurable so that new sensing applications can be implemented by the system 10 when new hardware sensing subsystems or new software processing algorithms are added to the system 10. Here, the clinician can configure the central hub 18 so that the monitoring of specific clinical applications can be added, deleted, or modified. The clinician is also able to configure the parameters of each sensor sub-system 30 through the central hub 18. This involves changing the sensitivity thresholds, sensing frequency, analysis procedures, or processing algorithms.

Still referring to FIG. 8, each sensing sub-system 30 can be considered a module, whereby it can be added or removed from the system 10 without compromising the performance of the other modules in the system 10. When a new module is added, it can be integrated into the existing network and effectively perform its sensing functions in an integrated fashion with the other modules. Each new sensor module comes with a software upgrade pack to upload new processing capabilities into the central hub 18. Thus, the software platform of the central hub 18 is also modular-based, such that new upgrades can integrate with the existing features to enable a host of additional processing capabilities that utilize the integrated information. Further, each sensor sub-system 30 is connected to a central hub 18 through a wireless network 29. The sensors 12 are able to communicate directly with the subsystem 30, and the subsystems 30 are able to communicate directly with the central hub 18 of the system 10 over the wireless network 29. The central hub 18 of the system 10 is then able to connect wirelessly to an external network of clinicians.

The central hub 18 of the system 10 is configuring such that collected information from multiple sensors 12 and/or sensing sub-systems 30 is integrated and processed. This central hub 18 is able to evaluate and perform operations on the sensed data. The result of the processing may trigger an action, which includes but is not limited to: an alarm to the patient; an alert to the hospital or clinic; a treatment to the patient; a reminder to the patient; a report to the patient; a report to the hospital or clinic; and/or a report to an insurance agency.

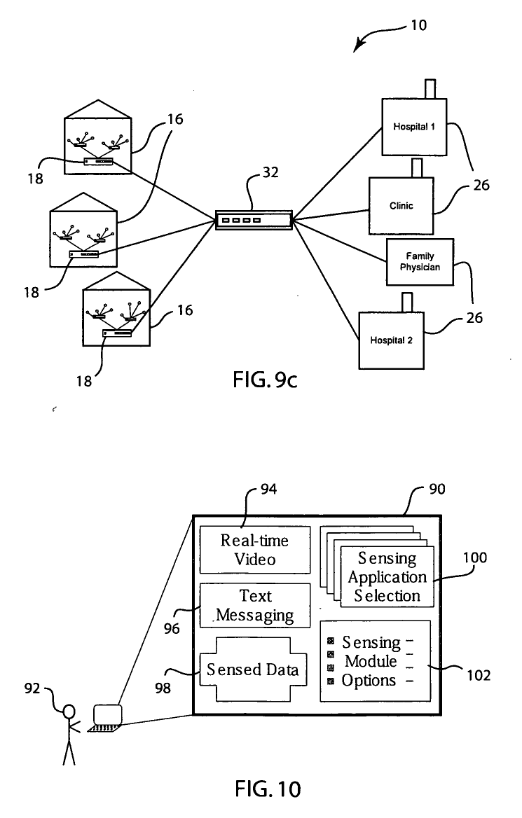

Referring to FIGS. 9a-9c, the system 10 also includes a network of clinicians 26 that monitor the information sent from the remote patient monitoring system 10. The clinicians 26 may include various configurations, as shown in the alternative embodiment of FIGS. 9a-9c. The central hub 18 may connect directly with a clinician 26 such as a hospital, as shown in FIG. 9a. Also, the central hub 18 may connect first to a local, and that routes or processes data from many central hubs 18 such as within a building, as shown in FIG. 9b. Alternatively, the central hub 18 may connect first to an external clinician hub 32, which processes and routes information from multiple home environments 16 to multiple clinicians 26, as shown in FIG. 9c. The clinicians 26 also provide a graphical user interface that the clinician can access to configure or re-configure the central hub 18 for new applications. This interface 90 may be through a web-based server. An example of an implementation of the interface 90 is shown in FIG. 10.

Referring to FIG. 10, the graphical user interface 90 of the present invention is accessed by a user 92, preferably through a web-based server. In a preferred embodiment, the graphical user interface 90 includes a real-time video interface 94, a text messaging interface 96, a sense data interface 98, a sensing application selection 100, and a sensing module options interface 102. In alternative embodiments, the graphical user interface 90 may be configured by the user 92 to meet the specific needs of the system 10 and the clinician 26.

The system 10 is to have secure data transfer by means of encryption, authentication, password registration, and permission control. The clinician is to have a password and be registered as an authorized user for the remote monitoring system of each patient for which clinician is providing care. The data being transferred between the remote patient and the clinician is not comprehensible by any third parties. The patient also has permission controls to modify only predetermined parameters of the remote sensing system.

The system 10 also includes secure data transfer by means of encryption, authentication, password registration, and permission control. The clinician 26 is to have a password and be registered as an authorized user 92 for the remote monitoring system 16 of each patient 14 for which clinician 26 is providing care. The data being transferred between the remote patient 14 and the clinician 26 is not comprehensible by any third parties. The patient 14 also has permission controls to modify only predetermined parameters of the remote sensing system.

The system 10 also includes a log file to record all prior system 10 operation. This log file may contain the patient's 14 sensed information, the configuration of the sensing subsystems 30, the configuration of the central hub 18, the communication transcript between the patient 14 and the clinician 26, the diagnosis by the clinician 26, and any notes by either the patient 14 or the clinician 26.

Finally, the remote patient monitoring system 10 is also configured to perform general error checking and monitor system 10 integrity. This includes monitoring power failures, system diagnostics, patient interference or tampering, and sensor performance. When an error is identified, or the system integrity is outside specifications, an alarm is generated to the clinician 26 and patient 14.

The present invention allows for a more practical and more efficient system for monitoring the health of remote patients. Since the system is configurable, the sensing applications can easily be modified depending on the changing conditions of the patient and the discretion of the clinician. When a patient develops a new medical condition, new sensing applications can easily be integrated into the existing remote sensing system. Unnecessary sensing applications can also be removed from the sensing system based on the needs of the patient. The modular nature of the proposed system allows for this flexibility of sensing applications and parameters. The remote sensing system also provides several levels of processing capabilities, so that data received from the sensors can be pre-processed at the sensing sub-system, processed at the home processing hub, and further analyzed at the clinician hub before being sent to the hospital or clinician. This processing is configurable to the specific needs of each patient and can integrate data from multiple sensing sub-systems. The result is that multiple sensing parameters can be analyzed in relation with each other to provide an integrated and case-specific analysis, all while the patient remains at the home site.

The present invention has been described in terms of specific embodiments incorporating details to facilitate the understanding of the principals of construction and operation of the invention. Such reference herein to specific embodiments and details thereof is not intended to limit the scope of the claims appended hereto. It will be apparent to those skilled in the art that modifications maybe made in the embodiment chosen for illustration without departing from the spirit and scope of the invention.

I claim:

1. A modular sensing system for remotely monitoring patient healthcare characteristics, the system comprising:
a plurality of sensors configured in a home environment of a patient, and further configured to collect a set of patient health characteristics from a patient, wherein each of the plurality of sensors is capable of sensing multiple parameters;
a central hub configured in the home environment, wherein the central hub receives the set of patient health characteristics, whereby the central hub is also configured to process the set of patent health characteristics and output a filtered set of patient health characteristics;
a wireless network configured to couple the plurality of sensors and the central hub, wherein each of the plurality of sensors is configured to continuously communicate with each other and with the central hub;
wherein each of the plurality of sensors is autonomous, thereby capable of automatic configuration with the modular sensing system.

2. The system as claimed in claim 1, wherein the size of the plurality of sensors are in a range from micro-scale to millimeter-scale.

3. The system as claimed in claim 2, wherein the plurality of sensors may be any of a number of sensor types including: invasive implantable; non-invasive, portable device embeddable; and, non-invasive, home device embeddable.

4. The system as claimed in claim 1, wherein each of the plurality of sensors are automatically configurable in real time.

5. The system as claimed in claim 1, wherein the plurality of sensors are portable and disposable.

6. The system as claimed in claim 1, wherein the central hub is further configured to evaluate the set of patient health characteristics as best, comparative or cumulative.

7. The system as claimed in claim 1, wherein each of the plurality of sensors is directly coupled with the central hub through the wireless network.

8. The system as claimed in claim 1, wherein the plurality of sensors are coupled with the central hub through the wireless network in an ad-hoc fashion.

9. The system as claimed in claim 1, further comprising a sensing sub-system configured to receive the set of patient health characteristics from the plurality of sensors, and further configured to relay the set of patient health characteristics to the central hub.

10. The system as claimed in claim 1, further comprising a clinician hub coupled with the central hub, the clinician hub configured to receive a patient status and the filtered set of patient health characteristics from the central hub and to send the patient status and the filtered set of patient health characteristics to an appropriate clinician.

11. The system as claimed in claim 1, wherein the central hub is configured to integrate multiple sensing parameters and multiple sensing applications.

12. A method of remotely monitoring patient healthcare characteristics, the method comprising:
collecting a set of patient healthcare characteristics from a patient with a plurality of sensors;
processing the set of patient healthcare characteristics in a central hub to produce a filtered set of patient health characteristics, wherein the plurality of sensors and the central hub are configured in a home environment, and further wherein the plurality of sensors send a patient status and the filtered set of patient health characteristics from the central hub to a clinician hub;
directing the patient status and the filtered set of patient health characteristics with the clinician hub to an appropriate clinician; and
accessing the patient status by the clinician with a graphical user interface.

13. The method as claimed in claim 12, wherein the size of the plurality of sensors are in a range from micro-scale to millimeter-scale.

14. The method as claimed in claim 13, wherein the plurality of sensors may be any of a number of sensor types including: invasive implantable; non-invasive, portable device embeddable; and, non-invasive, home device embeddable.

15. The method as claimed in claim 12, wherein the clinician hub is configured outside of the home environment.

16. The method as claimed in claim 12, wherein the patient status includes a report when the set of patient health characteristics is within a predetermined normal range, and the patient status includes the report and an alarm when the set of patient health characteristics are not in the predetermined normal range.

17. The method as claimed in claim 16, further comprising sending the alarm to the patient.

18. The method as claimed in claim 12, further comprising evaluating the set of patient health characteristics as best, comparative or cumulative with the central hub.

19. The method as claimed in claim 12, wherein each of the plurality of sensors is directly coupled with the central hub through the wireless network.

20. The method as claimed in claim 12, wherein the plurality of sensors are coupled with the central hub through the wireless network in an ad-hoc fashion.

21. The method as claimed in claim 12, further comprising receiving the set of patient health characteristics from the plurality of sensors with a sensing sub-system, and further comprising relaying the set of patient health characteristics to the central hub with the sensing sub-system.

22. The method as claimed in claim 12, further comprising integrating multiple sensing parameters and multiple sensing applications in the central hub.

23. A system for remotely monitoring patient healthcare characteristics, the system comprising:
a plurality of sensors configured in a home environment of a patient, and further configured to collect a set of patient health characteristics from a patient;
a central hub configured in the home environment, wherein the central hub receives the set of patient health characteristics, whereby the central hub is also configured to proceed the set of patient health characteristics and output a filtered set of patient health characteristics;
a wireless network configured to coupled the plurality of sensors and the central hub;
a clinician hub coupled with the central hub, the clinician hub configured to receive a patient status and the filtered set of patient health characteristics from the central hub and to send the patient status and the filtered set of patient health characteristics to an appropriate clinician; and
a graphical user interface configured such that the clinician can access the clinician hub and reconfigure the central hub.

24. The system as claimed in claim 23, wherein the size of the plurality of sensors are in a range from micro-scale to millimeter-scale.

25. The system as claimed in claim 24, wherein the plurality of sensors may be any of a number of sensor types including: invasive implantable; non-invasive, portable device embeddable; and, non-invasive, home device embeddable.

26. The system as claimed in claim 23, wherein the clinician hub is configured outside of the home environment.

27. The system as claimed in claim 23, wherein the patient status includes a report when the set of patient health characteristics is within a predetermined normal range, and the patient status including the report and an alarm when the set of patient health characteristics are not in the predetermined normal range.

28. The system as claimed in claim 27, wherein the alarm is also sent to the patient.

29. The system as claimed in claim 23, wherein the central hub is further configured to evaluate the set of patient health characteristics as best, comparative or cumulative.

30. The system as claimed in claim 23, wherein each of the plurality of sensors is directly coupled with the central hub through the wireless network.

31. The system as claimed in claim 23, wherein the plurality of sensors are coupled with the central hub through the wireless network in an ad-hoc fashion.

32. The system as claimed in claim 23, further comprising a sensing sub-system configured to receive the set of patient health characteristics from the plurality of sensors, and further configured relay the set of patient health characteristics to the central hub.

33. The system as claimed in claim 22, wherein the clinician reconfigures the central hub to alter the set of patient health characteristics collected by the plurality of sensors.

34. The system as claimed in claim 23, wherein the central hub is configured to integrate multiple sensing parameters and multiple sensing applications.
